(19) United States
(12) Patent Application Publication
(10) Pub. No.: US 2024/0083849 A1
(43) Pub. Date: Mar. 14, 2024

(54) NEW BISPHENOLS BEARING PENDANT CLICKABLE NORBORNENYL GROUP AND POLYMERS THEREFROM

(71) Applicant: COUNCIL OF SCIENTIFIC & INDUSTRIAL RESEARCH, New Delhi (IN)

(72) Inventors: Deepak Mannusing MAHER, Pune (IN); Samadhan Suresh NAGANE, Pune (IN); Prakash Purushottam WADGAONKAR, Pune (IN)

(21) Appl. No.: 18/259,365

(22) PCT Filed: Dec. 29, 2021
(86) PCT No.: PCT/IN2021/051214

§ 371 Date: Jun. 26, 2023

(30) Foreign Application Priority Data
Dec. 30, 2020 (IN) 202011057103

(51) Int. Cl. C07D 209/46; C08G 63/685; C08G 63/81

(52) U.S. Cl. CPC C07D 209/46; C08G 63/6856; C08G 63/81

(57) ABSTRACT

The present invention relates to a new bisphenol bearing pendant clickable norbornenyl group of formula (I) and its aromatic polyesters bearing pendant norbornenyl groups of formula (II) and processes for the preparation thereof.

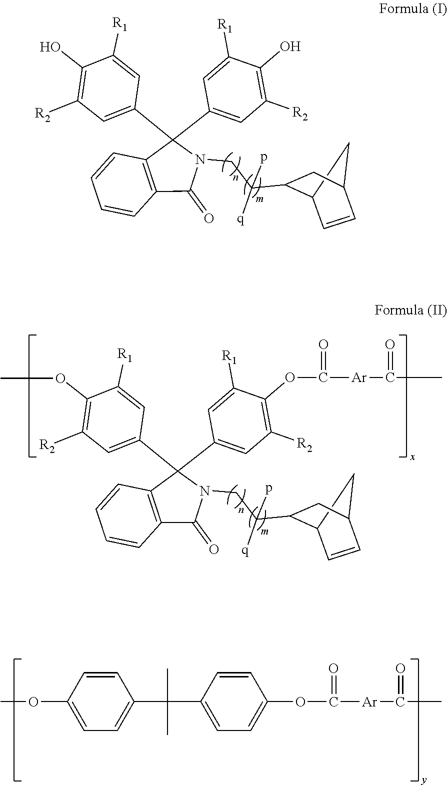

### FIELD OF THE INVENTION

The present invention relates to a new bisphenol bearing pendant clickable norbornenyl group and polymers therefrom. More particularly, the present invention relates to a new bisphenol bearing pendant clickable norbornenyl group of formula (I) and aromatic polyesters bearing pendant norbornenyl groups of formula (II) and processes for the preparation thereof.

### BACKGROUND AND PRIOR ART OF THE INVENTION

Bisphenols are the building blocks for a range of industrially relevant polymers such as aromatic polycarbonates, polyesters, poly(arylene ether)s, cyanate esters, epoxy resins, etc. Therefore, there is a strong interest in the design and synthesis of new bisphenols with special structural features. Bisphenols containing pendant (clickable) functional groups are particularly attractive as they provide access to step-growth polymers fitted with pendant reactive functional groups which could subsequently be post-modified with appropriate reagents. Bisphenols possessing pendant clickable functional groups such as open chain alkene, propargyloxy, azido, furyl or maleimide are known in the literature. However, bisphenols bearing pendant cyclo-alkene group such as norbornenyl are scarce except for 1,4-dihydro-1,4-methanonaphthalene-5,8-diol.

Polymers possessing pendant or end-standing norbornenyl group(s) are valuable precursors for various macromolecular architectures such as graft copolymers, block copolymers, cross-link polymers, etc as reactive alkene functional group in norbornenyl moiety is capable of undergoing a set of interesting click reactions such as tetrazine-norbornene inverse electron demand Diels-Alder reaction, photoinitiated thiol-norbornene reaction, metal-free azide-norbornene 1,3-dipolar cycloaddition reaction and so on. Polymers bearing pendant norbornenyl groups have been synthesized via the following routes: A) ring-opening polymerization of cyclic monomers containing norbornenyl group B) step growth polymerization of monomers bearing pendant norbornenyl group and C) chemical modification of preformed polymers using appropriate norbornenyl-containing reagents.

Therefore, there is a need in the art to provide such bisphenol bearing pendant norbornenyl groups and their polymers. There is also a need to provide cheap, simple and industrially applicable process for the preparation of these compounds.

### OBJECTS OF THE INVENTION

The main objective of the present invention is to provide a new bisphenol bearing pendant clickable norbornenyl group of formula (I) and process for the preparation thereof.

Yet another objective of the present invention is to provide aromatic polyesters bearing pendant norbornenyl groups of formula (II) from a new bisphenol bearing pendant clickable norbornenyl group and process for the preparation thereof.

Still another objective of the present invention is to provide a post-modified polymer of polymers possessing pendant norbornenyl groups of formula (II) and process for the preparation thereof.

### SUMMARY OF THE INVENTION

Accordingly, to accomplish the objectives, the present invention provides a new bisphenol bearing pendant clickable norbornenyl group of formula (I) and its aromatic polyesters bearing pendant norbornenyl groups of formula (II), and processes for the preparation thereof.

In an embodiment, the present invention provides a new bisphenol bearing pendant clickable norbornenyl group of formula (I) below:

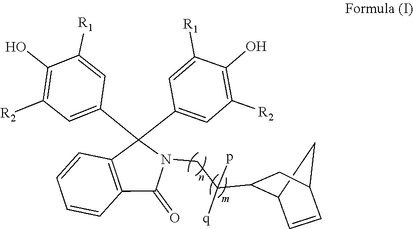

wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6.

Another embodiment of the present invention provides a process for the preparation of a new bisphenol bearing pendant clickable norbornenyl group of formula (I), wherein said process comprises of stirring the reaction mixture comprising of primary alkyl amine possessing norbornenyl, compound (2), and phenolphthalein derivative, compound (1), in the molar ratio ranging between 10:1 to 25:1 for a period in the range of 30 to 60 h at a temperature in the range of 120° C. to 160° C. to afford bisphenol of formula (I).

One more embodiment of the present invention provides aromatic polyesters bearing pendant norbornenyl groups of formula (II) from a new bisphenol bearing pendant clickable norbornenyl group of formula (I).

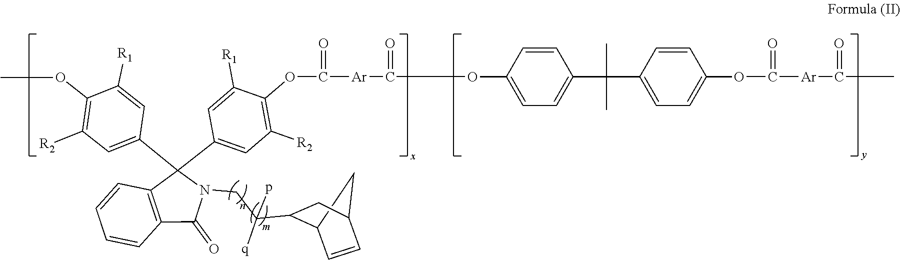

wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6;
wherein, x=0.1-1, and y=0-0.9;
Ar=isophthaloyl chloride, terephthaloyl chloride, or mixtures thereof.

Yet another embodiment of the present invention provides a process for the preparation of polyester of formula (II), wherein said process comprises the steps of:
a) dissolving monomer of formula (I) and/or bisphenol A in an alkali solution and stirring the reaction mixture;
b) adding benzyltriethylammonium chloride to the reaction mixture of step (a) and stirring followed by addition of a solution of diacid chloride in dichloromethane to the reaction mixture and stirring vigorously; and
c) pouring the reaction mixture of step (b) into hot water; filtering the precipitated polymer and washing it several times with water followed by work-up to afford polyester of formula (II).

In another embodiment of the present invention, polymers of formula (II) possessing pendant norbornenyl groups are capable of undergoing tetrazine-norbornene inverse electron demand Diels-Alder reaction, photoinitiated thiol-norbornene reaction or metal-free azide-norbornene 1,3-dipolar cycloaddition click reaction to form post-modified polymers. Polymers bearing pendant norbornenyl groups of formula (II) could be cross-linked thermally or photochemically.

### BRIEF DESCRIPTION OF THE DRAWINGS

FIG. 1: illustrates ¹H-NMR spectrum (in DMSO-d₆) of 2-(bicycle [2.2.1] hept-5-en-2-ylmethyl) 3,3-bis (4-hydroxyphenyl) isoindolin-1-one, (Monomer-1)

FIG. 2: illustrates ¹H-NMR spectrum (in CDCl₃) of aromatic polyester, (polymer-1), obtained by polycondensation of 2-(bicycle [2.2.1] hept-5-en-2-ylmethyl) 3,3-bis (4-hydroxyphenyl) isoindolin-1-one with isophthaloyl chloride.

FIG. 3: illustrates ¹H-NMR spectra of copolyester bearing pendant norbornenyl groups (polymer-2). (a) before post-modification, (b) after post modification via norbornene-tetrazine click reaction

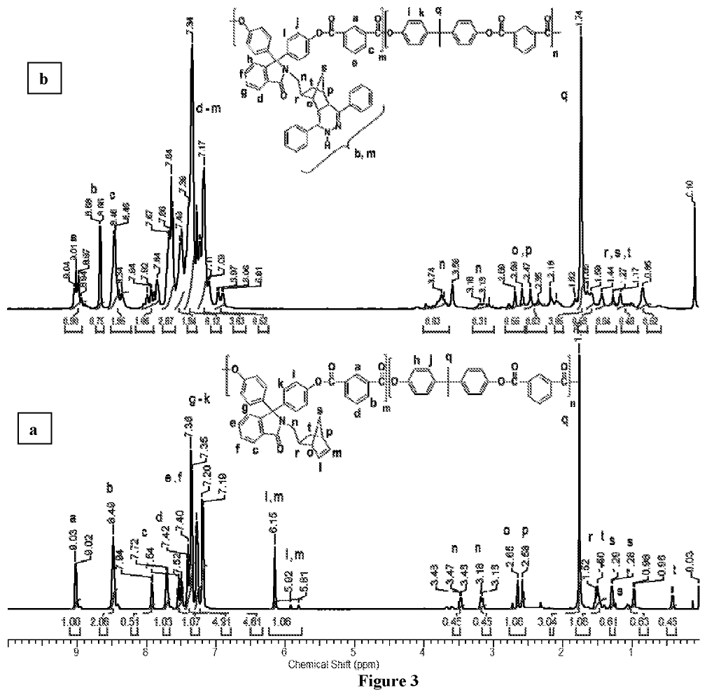

FIG. 4: (a): illustrates reaction of copolyester polymer-2 dissolved in DMF with 25 mol % PETMP in the presence of DMPA (5 mol % relative to thiol) as a photoinitiator. (b): illustrates exposure of the reaction mixture to 365 nm light at a temperature in the range of 25-30° C. for 1 minute when the homogeneous solution turned into a cross-linked polymer.

### DETAILED DESCRIPTION OF THE INVENTION

It is to be understood that the figures and descriptions of the present invention have been simplified to illustrate elements that are relevant for a clear understanding of the invention. The detailed description will be provided herein below with reference to the attached drawing.

The present invention provides a new bisphenol bearing pendant clickable norbornenyl group of formula (I) and its aromatic polyesters bearing pendant norbornenyl groups of formula (II), and processes for the preparation thereof.

In an embodiment, the present invention provides a new bisphenol bearing pendant clickable norbornenyl group of formula (I) below:

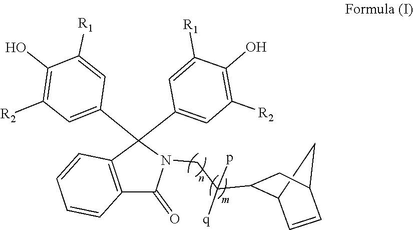

wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6.

Another embodiment of the present invention provides a process for the preparation of a new bisphenol bearing pendant clickable norbornenyl group of formula (I), wherein said process comprises of stirring the reaction mixture comprising of primary alkyl amine possessing norbornenyl group, compound (2), and phenolphthalein derivative, compound (1), in the molar ratio ranging between 10:1 to 25:1 for the period in the range of 30 to 60 hours at a temperature in the range of 120° C. to 160° C. to afford bisphenol of formula (I). The general process depicted below in scheme-1:

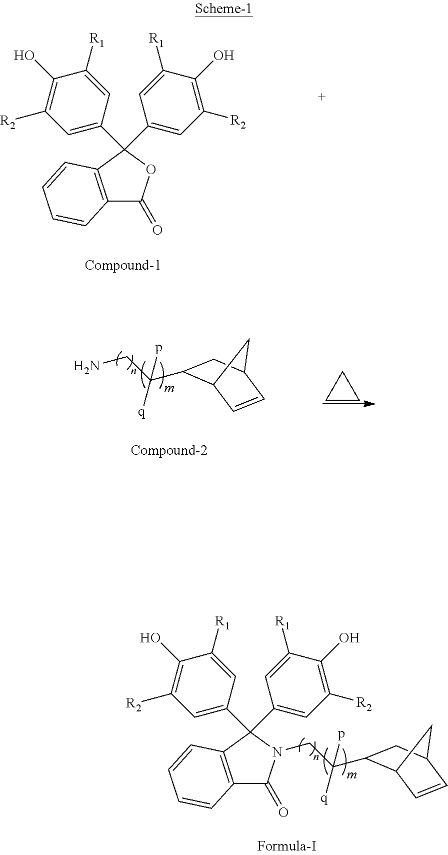

wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6.

One more embodiment of the present invention provides an aromatic polyester bearing pendant norbornenyl groups of formula (II) from a new bisphenol bearing pendant clickable norbornenyl group of formula (I).

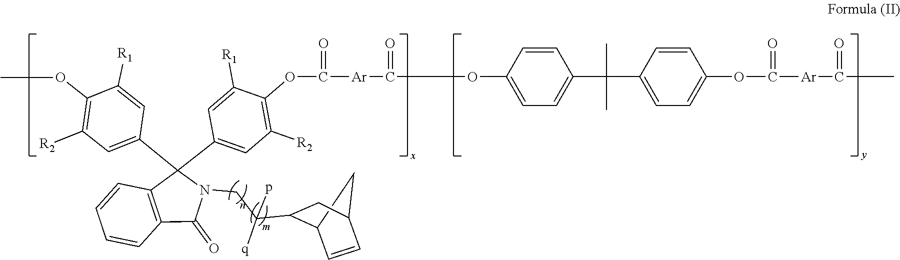

wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6;
wherein, x=0.1-1, and y=0-0.9;
Ar=isophthaloyl chloride, terephthaloyl chloride, or mixtures thereof.

Yet another embodiment of the present invention provides a process for the preparation of polyester of formula (II), wherein said process comprises the steps of:
a) dissolving monomer of formula (I) and/or bisphenol A in an alkali solution and stirring the reaction mixture;
b) adding benzyltriethylammonium chloride to the reaction mixture of step (a) and stirring followed by addition of a solution of diacid chloride in dichloromethane to the reaction mixture and stirring vigorously; and
c) pouring the reaction mixture of step (b) into hot water; filtering the precipitated polymer and washing it several times with water followed by work-up to afford polyester of formula (II).

Diacid chloride is selected from isophthaloyl chloride, terephthaloyl chloride, or mixtures thereof.

The general process for the preparation of polyester of formula (II) is depicted below in scheme-2:

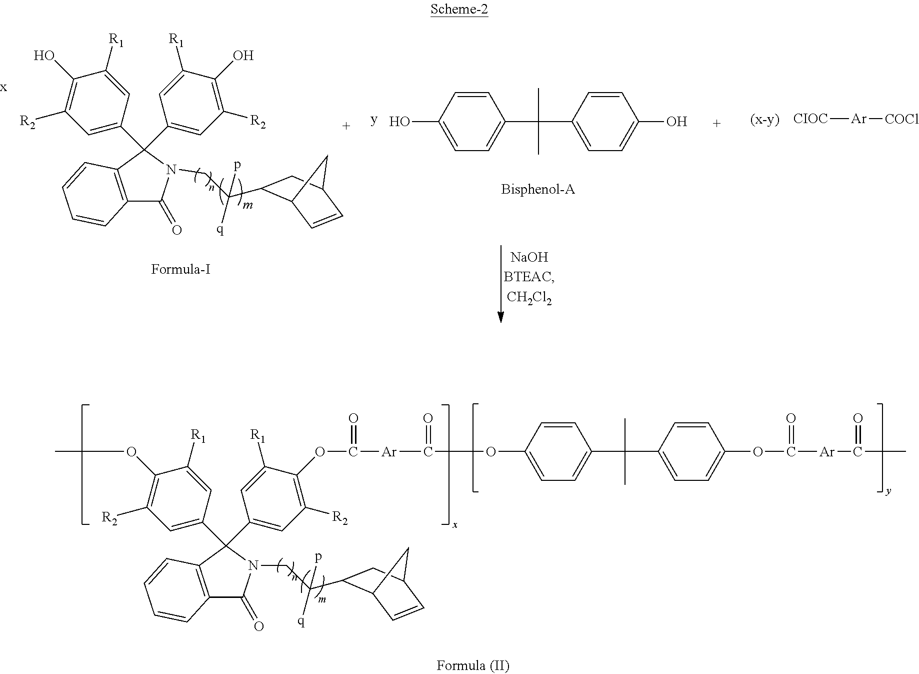

wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6;
wherein, x=0.1-1, and y=0-0.9;
Ar=isophthaloyl chloride, terephthaloyl chloride, or mixtures thereof.

In another embodiment of the present invention, polymers of formula (II) possessing pendant norbornenyl groups are capable of undergoing tetrazine-norbornene inverse electron demand Diels-Alder reaction, photoinitiated thiol-norbornene reaction or metal-free azide-norbornene 1,3-dipolar cycloaddition click reaction to form post-modified polymers. Polymers bearing pendant norbornenyl groups of formula (II) could be cross-linked thermally or photochemically. The highly reactive strained ring of norbornenyl group provides a bundle of opportunities for post-modifications. For instance, norbornenyl group is capable of reacting with azides by heat-promoted 1,3-dipolar cycloaddition, the inverse electron demand Diels-Alder reaction with tetrazine and radical addition of thiols promoted by UV irradiation.

In preferred embodiment of the present invention, a representative copolyester bearing pendant norbornenyl groups are post-modified with tetrazine viz., 3,6-diphenyl-1,2,4,5-tetrazine via inverse electron demand Diels-Alder reaction. Norbornene-tetrazine click reaction is utilized as a tool for chemical modification. A representative copolyester bearing pendant norbornenyl groups polymer-2 was reacted with 1.5 equivalents of 3,6-diphenyl-1,2,4,5-tetrazine at a temperature in the range of 25-30° C. for 6 hours in dichloromethane as a solvent to get modified polymer of formula (A). Reaction is depicted below in scheme-4.

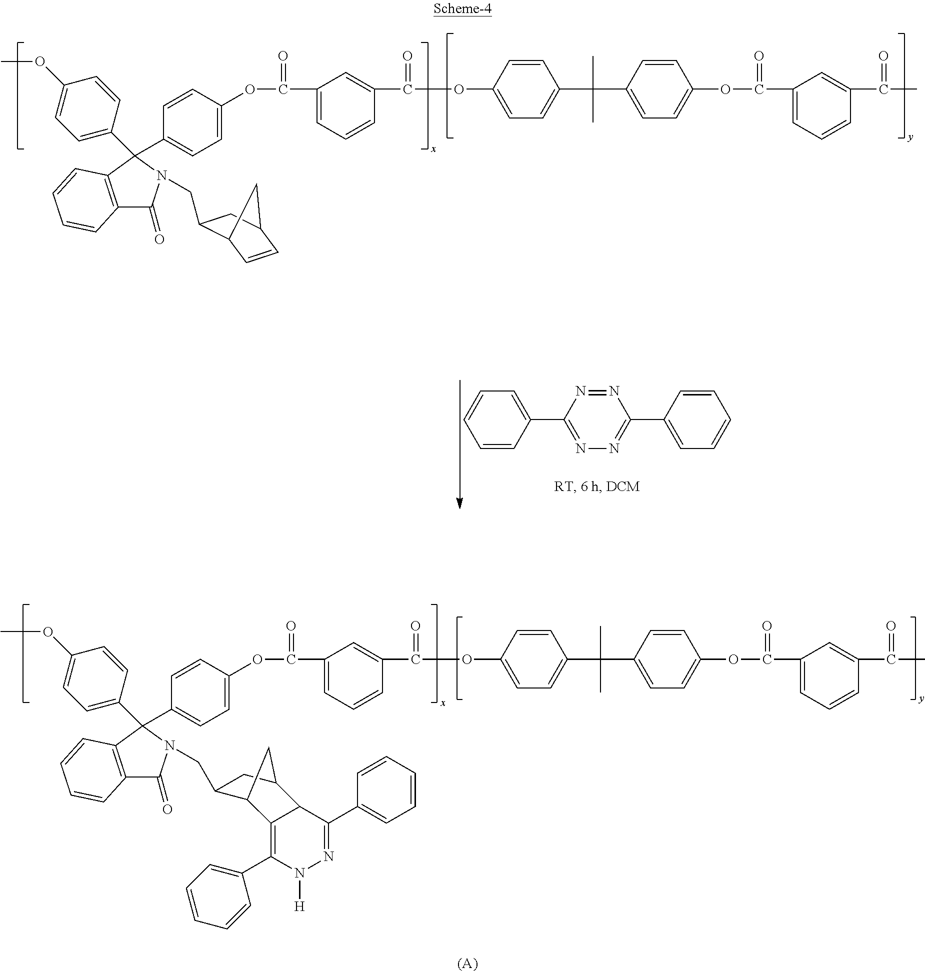

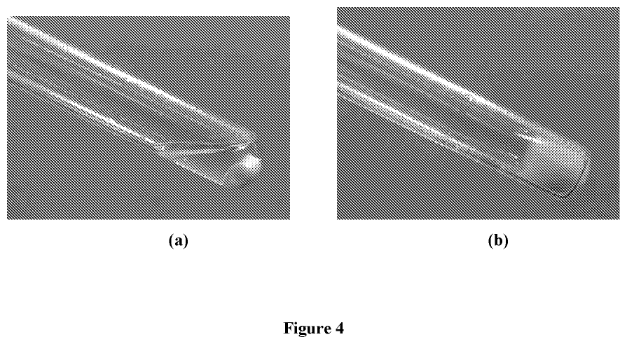

After successful post-modification of aromatic copolyester via norbornene-tetrazine click reaction, the efficacy of reaction of pendant norbornenyl groups towards norbornene-thiol click reaction is investigated using a multifunctional thiol as shown in Scheme-5. In a representative example, copolyester, polymer-2, dissolved in DMF was reacted with 25 mol % tetrakis (3-mercapto propionate) (PETMP) in the presence of DMPA (5 mol % relative to thiol) as a photoinitiator (FIG. 4(a)). The reaction mixture was exposed to 365 nm light at a temperature in the range of 25-30° C. for 1 minute when the homogeneous solution turned into a cross-linked polymer (B) as depicted in FIG. 4(b).

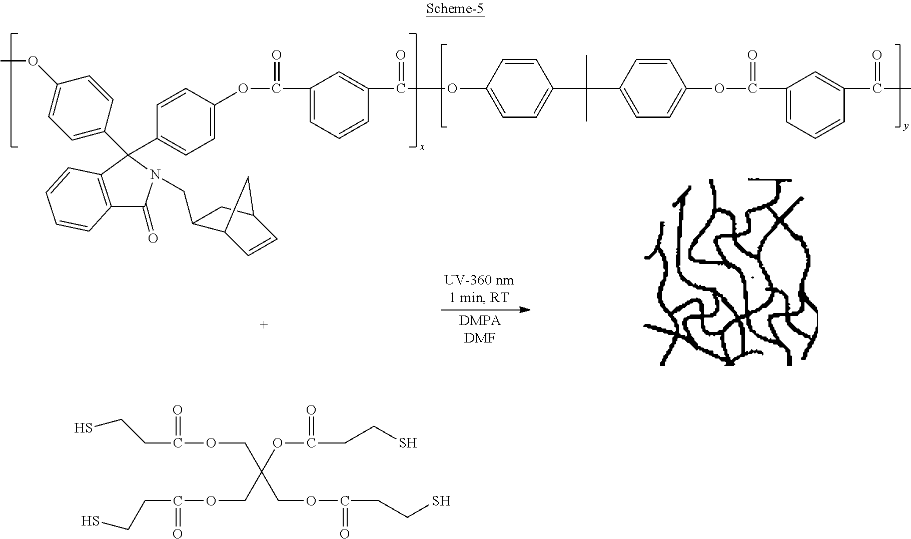

### EXAMPLES

Following examples are given by way of illustration and therefore should not be construed to limit the scope of the invention.

### Example 1: General Process for the Preparation of Bisphenol of Formula (I)

Into a 250 mL two-necked round bottom flask equipped with a reflux condenser were charged phenolphthalein derivative of compound (1) (1.0 eq) and compound of compound (2) (a mixture of endo and exo isomers 80:20 mol %) (12.0 eq) and the reaction mixture was heated under stirring at 150° C. for 48 hours. The excess compound of compound (2) was removed under reduced pressure. The reaction mixture was cooled to 30° C. and dissolved in ethyl acetate and the solution was washed with water. The ethyl acetate layer was separated, dried over anhydrous sodium sulfate, filtered and ethyl acetate was removed under reduced pressure at 30° C. The crude product was dissolved in aqueous sodium hydroxide solution and the solution was acidified with dilute hydrochloric acid. The precipitated product was separated out by filtration and washed with cold water. The product was recrystallized from a mixture of ethanol:water (1:1, v/v) to afford of bisphenol of formula (I).

### Monomer-1: 2-(bicycle [2.2.1] hept-5-en-2-ylmethyl)-3,3-bis (4-hydroxyphenyl) isoindolin-1-one (BPN)

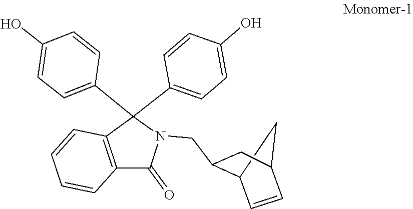 [SMILES: O=C1c2ccccc2C(c2ccc(O)cc2)(c2ccc(O)cc2)N1CC1CC2C=CC1C2]

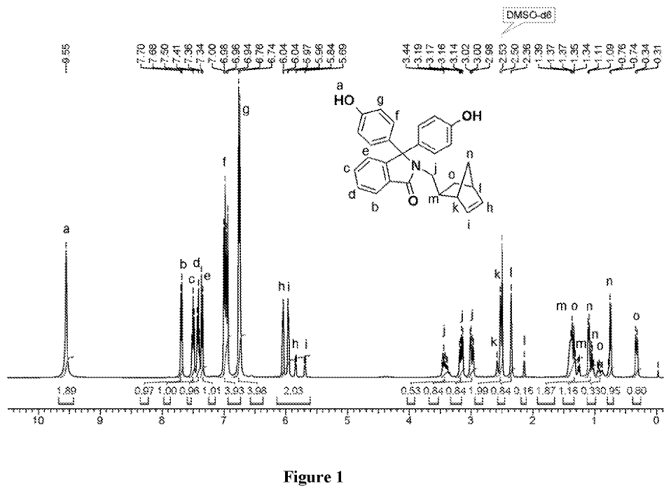

White crystals: Yield: 10.65 g (80%); Melting point: >300° C.; FT-IR (KBr, cm⁻¹): 3385, 2960, 1636, 1502; ¹H NMR (400 MHz, DMSO-d₆, δ/ppm) (FIG. 1)=9.55 (br. s., 2H), 7.69 (d, 1H), 7.50 (t, 1H), 7.41 (t, 1H), 7.35 (d, 1H), 7.00-6.94 (m, 4H), 6.75 (d, 4H), 6.06-5.68 (m, 2H), 3.48-2.97 (m, 2H), 2.55 (d, 2H), 2.36-2.15(d, 1H), 1.39-1.25 (m, 2H), 1.11-1.02 (m, 1H), 0.93-0.74 (m, 1H), 3.33 (d, 1H); ¹³C NMR (DMSO-d₆, δ/ppm): 167.8, 167.4, 157.2, 157.0, 151.9, 136.8, 136.4, 136.2, 132.8, 132.1, 130.9, 130.7 130.2, 130.1, 129.5, 129.3, 129.2, 129.0, 127.8, 132.5, 122.7, 115.3, 74.7, 49.1, 44.8, 44.5, 44.0, 41.9, 41.1, 37.6, 37.2, 30.6.

### Example 2: General Process for the Preparation of Polyester of Formula (II)

An oven-dried, 100 mL two-necked round bottom flask equipped with a high-speed mechanical stirrer and an addition funnel was charged with formula (I) (1.0 eq) and 1 M aqueous solution of sodium hydroxide (2.0 eq). The reaction mixture was stirred at 10° C. for 1 hour. Thereafter, benzyltriethylammonium chloride (BTEAC) (0.06 eq) was added to the reaction mixture. The solution of isophthaloyl chloride (1.0 eq) dissolved in dichloromethane was added in one lot to the reaction mixture and the mixture was stirred vigorously at 2000 rpm for 1 hour. After completion of reaction time, the reaction mixture was poured into hot water, the precipitated polymer was filtered and washed with water. Polymer was dried and dissolved in dichloromethane and reprecipitated into mixture of methanol: water (1:1, v/v). The fibrous polymer was filtered, washed with methanol and dried at 50° C. under reduced pressure for 24 h.

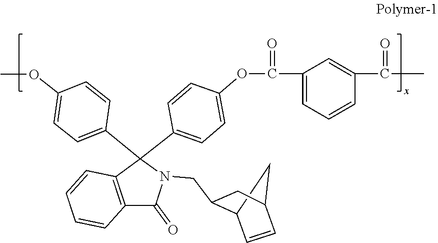

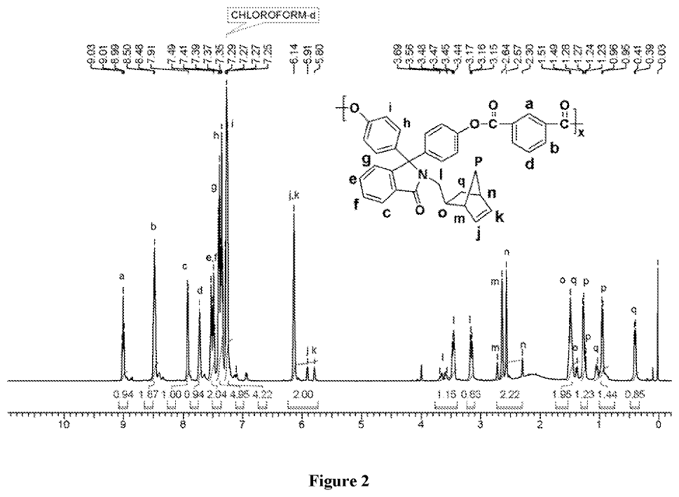

FT-IR (CHCl₃, cm⁻¹): 2960, 1740, 1685, 1502; ¹H NMR (400 MHz, CDCl₃, δ/ppm) (FIG. 2)=9.01 (t, 1H), 8.49 (t, 1H), 7.93 (d, 1H), 7.74-7.71 (m, 1H), 7.55-7.48 (m, 2H), 7.41-7.34 (m, 5H), 7.27-7.25 (m, 4H), 6.14-6.50 (m, 2H), 3.67-3.15 (m, 2H), 2.72-2.30 (m, 2H), 1.51-1.38 (m, 2H), 1.29-1.24 (m, 1H), 1.06-0.95 (m, 2H), 0.40 (d, 1H), ¹³C NMR (CDCl₃, δ/ppm):168.7, 164.0, 150.7, 150.5, 150.4, 138.5, 138.3, 137.4, 135.1, 132.9, 132.3, 131.8, 130.7, 130.0, 129.8, 129.2, 128.5, 123.8, 123.3, 121.9, 75.1, 49.5, 45.6, 45.1, 42.4, 38.3, 31.1.

### Example 3: Representative Process for the Preparation of Copolyester (50:50) of Formula (II) with using Bisphenol-A as a Comonomer

An oven-dried, 100 mL two-necked round bottom flask equipped with a high-speed mechanical stirrer and an addition funnel was charged with formula (I) (0.5) eq), bisphenol-A (0.5 eq) and 1 M aqueous solution of sodium hydroxide (2.0 eq). The reaction mixture was stirred at 10° C. for 1 hour. Thereafter, benzyltriethylammonium chloride (BTEAC) (0.06 eq) was added to the reaction mixture. The solution of isophthaloyl chloride (1.0 eq) dissolved in dichloromethane was added in one lot to the reaction mixture and the mixture was stirred vigorously at 2000 rpm for 1 hour. After completion of reaction time, the reaction mixture was poured into hot water, the precipitated polymer was filtered and washed with water. Polymer was dried and dissolved in dichloromethane and reprecipitated into mixture of methanol: water (1:1, v/v). The fibrous copolymer was filtered, washed with methanol and dried at 50° C. under reduced pressure for 24 hours. The copolyester is designated as polymer-2.

Copolyesters were synthesized by polycondensation of varying molar ratios of monomer-1 and BPA with IPC using the similar procedure. Copolyesters were designated as polymer-3 and polymer-4 wherein the feed % molar composition of monomer-1 and BPA was 30:70, and 10:90, respectively.

### Synthesized Co-Polymer

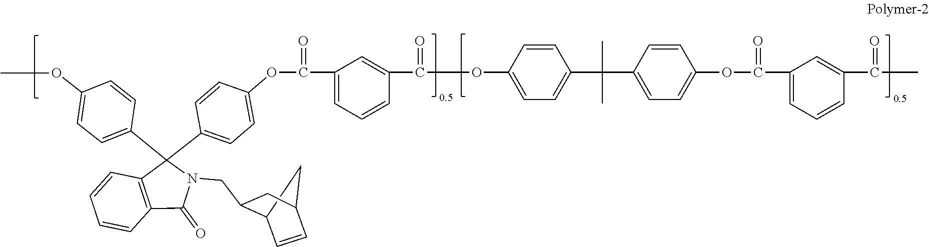

### Example 4: Post-Modification of Polymer-2 Via Tetrazine-Norbornene Click Reaction

In an oven-dried Schlenk tube were placed polymer-2 (100 mg, 2.19×10−3 mol), 3,6-diphenyl-1,2,4,5-tetrazine (103 mg, 4.39×10−3 mol) and dry DCM (1 mL). The reaction mixture was degassed by purging with nitrogen and stirred at room temperature for 6 hours. The reaction mixture was poured into methanol (25 mL) to precipitate the polymer. The precipitated polymer was filtered off and then dried under vacuum at room temperature for 8 h. The post-functionalized polymer was designated as polymer-2-Tz.

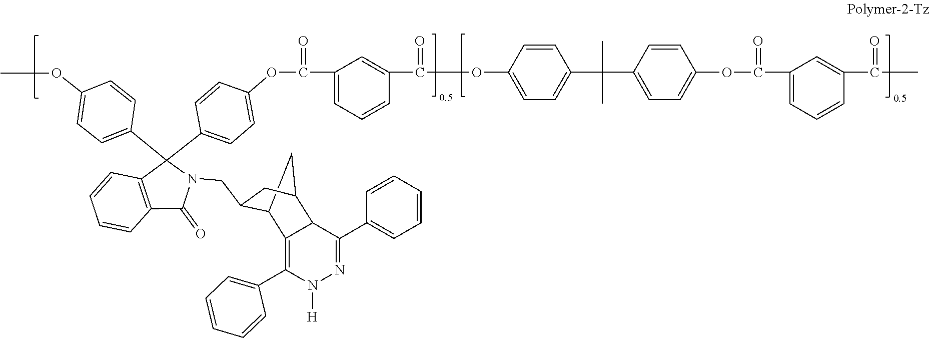

### Example 5: Cross-Linking of Copolyester, Polymer-2, Via Thiol-Norbornene Click Reaction

In an oven-dried Schlenk tube were placed polymer-2 (100 mg, 2.19×10⁻³ mol), tetrakis (3-mercapto propionate (30 mg, 0.657×10⁻³ mol), DMPA (5 mol % relative to thiol) and dry N,N-dimethyl formamide (1 mL). The reaction mixture was degassed by purging with nitrogen and then irradiated using a UV lamp at 365 nm for 1 minute to obtain the cross-linked polymer.

### ADVANTAGES OF THE INVENTION

Bisphenols bearing pendant norbornenyl groups were readily synthesized starting from commercially available phenolphthalein via single-step reaction with primary alkyl amines possessing norbornenyl moiety
These cardo bisphenols are potentially useful for the synthesis of aromatic polycarbonates, polyesters, poly(arylene ether)s, cyanate esters, epoxy resins, etc.
Polymers possessing pendant norbornenyl groups are capable of undergoing tetrazine-norbornene inverse electron demand Diels-Alder reaction, photoinitiated thiol-norbornene reaction or metal-free azide-norbornene 1,3-dipolar cycloaddition click reaction to form modified polymers
Polymers bearing pendant norbornenyl groups could be cross-linked thermally or photochemically.

We claim:

1. A new bisphenol bearing pendant clickable norbornenyl group of formula (I) below: 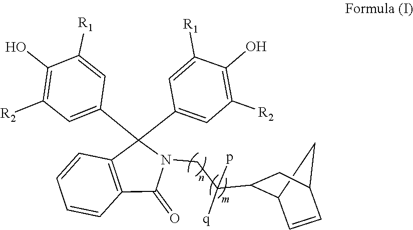
wherein, R₁ and R₂ are selected from a group comprising of H, C₁-C₅ substituted or unsubstituted, straight or branched alkyl, alkoxy;
p and q are selected form a group comprising of H and F;
m=0 or 1; n=0-6.

2. A process for the preparation of a new bisphenol bearing pendant clickable norbornenyl group of formula (I) as claimed in claim 1, wherein said process comprises of stirring the reaction mixture comprising of primary alkyl amine possessing norbornenyl group, compound (2), and phenolphthalein derivative, compound (1), in a molar ratio ranging between 10:1 to 25:1 for a period in a range of 30 to 60 hours at a temperature in a range of 120° C. to 160° C. to afford bisphenol of formula (I); 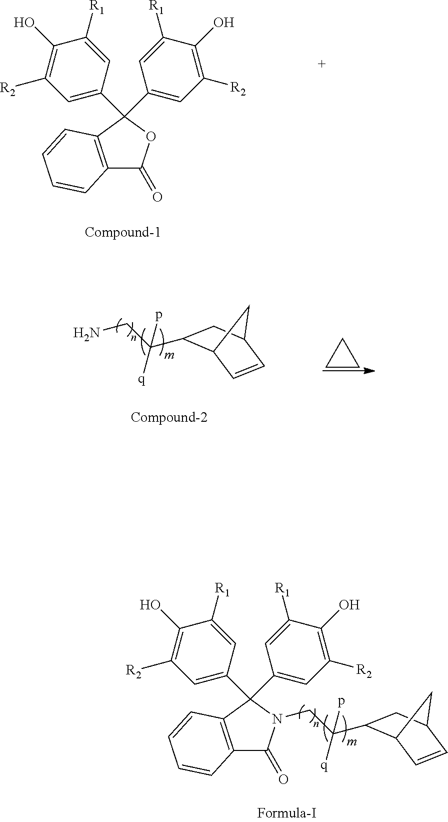
wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6.

3. An aromatic polyester bearing pendant norbornenyl groups of formula (II); 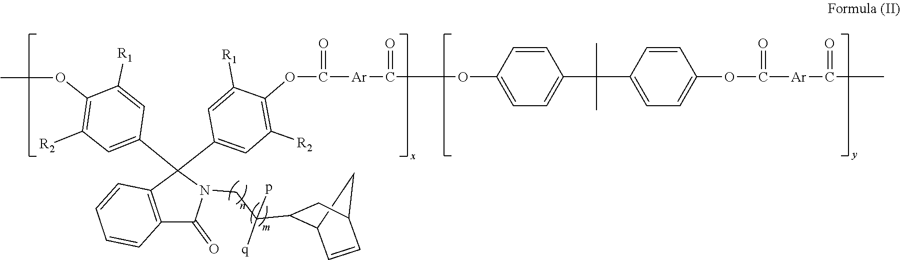
wherein, R₁ and R₂ are selected from a group comprising of H, C₁-C₅ substituted or unsubstituted, straight or branched alkyl, alkoxy;
p and q are selected form a group comprising of H and F;
5 m=0 or 1; n=0-6;
wherein, x=0.1-1, and y=0-0.9;
Ar=isophthaloyl chloride, terephthaloyl chloride, or mixtures thereof.

4. A process for the preparation of polyester of formula (II) as claimed in claim 3, wherein said process comprises the steps of:
a) dissolving monomer of formula (I) and bisphenol A in an alkali solution and stirring the reaction mixture;
b) adding benzyltriethylammonium chloride to the reaction mixture of step (a) and stirring followed by addition of a solution of diacid chloride in dichloromethane to the reaction mixture and stirring vigorously; and
c) pouring the reaction mixture of step (b) into hot water; filtering the precipitated polymer and washing it several times with water followed by work-up to afford polyester of formula (II). 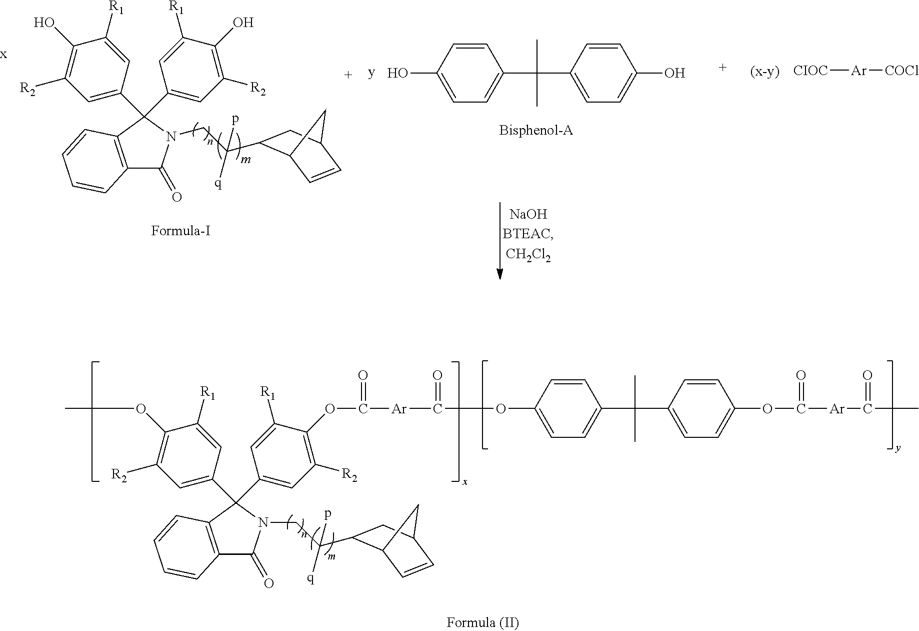
wherein, R₁ and R₂ are selected from the group comprising of H, C₁-C₅ substituted or unsubstituted, straight or branched alkyl, alkoxy;
p and q are selected form the group comprising of H and F;
m=0 or 1; n=0-6;
wherein, x=0.1-1, and y=0-0.9;
Ar=isophthaloyl chloride, terephthaloyl chloride, or mixtures thereof.

5. The process as claimed in claim 4, wherein said diacid chloride is selected from isophthaloyl chloride, terephthaloyl chloride, or mixtures thereof.

6. The aromatic polyester bearing pendant norbornenyl groups of formula (II) as claimed in claim 3, wherein said polymer is cross-linked thermally or photochemically.

7. The aromatic polyester bearing pendant norbornenyl groups of formula (II) as claimed in claim 3, wherein said polymer are capable of undergoing tetrazine-norbornene inverse electron demand Diels-Alder reaction, photoinitiated thiol-norbornene reaction or metal-free azide-norbornene 1,3-dipolar cycloaddition click reaction to form modified polymers.
